SEQUENCE LISTING (19 sequences)

SEQ ID NO: 1 - protein, 6 aa, Artificial Sequence
  Synthetic sequence (human CD28 partial sequence)
VYSKTG

SEQ ID NO: 2 - protein, 4 aa, Artificial Sequence
  Synthetic sequence (human CD28 partial sequence)
YSKT

SEQ ID NO: 3 - protein, 5 aa, Artificial Sequence
  Synthetic sequence (human CD28 partial sequence)
YSKTG

SEQ ID NO: 4 - protein, 5 aa, Artificial Sequence
  Synthetic sequence (human CD28 partial sequence)
VYSKT

SEQ ID NO: 5 - protein, 6 aa, Artificial Sequence
  Synthetic sequence (human CD28 partial sequence)
YSKTGF

SEQ ID NO: 6 - protein, 7 aa, Artificial Sequence
  Synthetic sequence (human CD28 partial sequence)
VYSKTGF

SEQ ID NO: 7 - protein, 5 aa, Artificial Sequence
  Synthetic sequence (human CD28 partial sequence)
SKTGF

SEQ ID NO: 8 - protein, 8 aa, Artificial Sequence
  Synthetic sequence (human CD28 partial sequence)
GNYSQQLQ

SEQ ID NO: 9 - DNA, 321 bp, Artificial Sequence
  Synthetic sequence (monoclonal antibody 9D7 partial sequence)
gatatccagacgacacagactacatcctcccgttctgcctctctgggagacagagtcaccatcagttgcagggcaggtcaggacattagtaattatttaaactggtatcagcagaaaccagatggaactgttaagctcctgatctactacacatcaagattacactcaggagtcccatcaaggttcagtggcagtgggtctggaacagattattctctcaccattagcaacctggagcaagaagatattgccacttacttttgccaacagggtcatacgcttccgtggacgttcggtggaggcaccaagctggaaatcaaa

SEQ ID NO: 10 - protein, 107 aa, Artificial Sequence
  Synthetic sequence (monoclonal antibody 9D7 partial sequence)
DIQTTQTTSSLSASLGDRVTISCRAGQDISNYLNWYQQKPDGTVKLLIYYTSRLHSGVPSRFSGSGSGTDYSLTISNLEQEDIATYFCQQGHTLPWTFGGGTKLEIK

SEQ ID NO: 11 - DNA, 363 bp, Artificial Sequence
  Synthetic sequence (monoclonal antibody 9D7 partial sequence)
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagatacagtggtagtactagctacaatccatctctcaaaagtcgaatctctatcactcgagacacatccaagaaccagttcttcctgcagttgaattctgtgactactgaggacacagccacatattactgtgcaagagattggccgcgaccgagctactggtacttcgatgtctggggcgcagggaccacggtcaccgtctcctca

SEQ ID NO: 12 - protein, 121 aa, Artificial Sequence
  Synthetic sequence (monoclonal antibody 9D7 partial sequence)
DVQLQESGPGLVKPSQSLSLTCTVTGYSITSDYAWNWIRQFPGNKLEWMGYIRYSGSTSYNPSLKSRISITRDTSKNQFFLQLNSVTTEDTATYYCARDWPRPSYWYFDVWGAGTTVTVSS

SEQ ID NO: 13 - DNA, 360 bp, Artificial Sequence
  Synthetic sequence (monoclonal antibody 5.11A partial sequence)
caggtccaactgcagcagtccggacctgagctggtgaagccggggacttcagtgaggatttcctgcgaggcttctggctacaccttcacaagctactatatacactgggtgaaacagaggcctggacagggacttgagtggattggatgtatttatcctggaaatgtcaatactaactataatgagaagttcaaggacaaggccacactgattgtagacacatcctccaacactgcctacatgcagctcagcagaatgacctctgaggactctgcggtctatttctgtacaagatcacactacggcctcgactggaacttcgatgtctggggcgcagggaccacggtcaccgtctcctca

SEQ ID NO: 14 - protein, 120 aa, Artificial Sequence
  Synthetic sequence (monoclonal antibody 5.11A partial sequence)
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 15 - DNA, 321 bp, Artificial Sequence
  Synthetic sequence (monoclonal antibody 5.11A partial sequence)
gacatccagatgaaccagtctccatccagtctgtctgcatcccttggagacacaattaccatcacttgccatgccagtcaaaacatttatgtttggttaaactggtaccagcagaaaccaggaaatattcctaaactcttgatctataaggcttccaacctgcacacaggcgtcccatcaaggtttagtggcagtggatctggaacaggcttcacattaaccatcagcagcctgcagcctgaagacattgccacttactactgtcaacagggtcaaacttatccgtacacgttcggaggggggaccaagctggaaataaaa

SEQ ID NO: 16 - protein, 107 aa, Artificial Sequence
  Synthetic sequence (monoclonal antibody 5.11A partial sequence)
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 17 - protein, 15 aa, Artificial Sequence
  Synthetic sequence (human CD28 partial sequence)
GNYSQQLQVYSKTGF

SEQ ID NO: 18 - protein, 107 aa, Artificial Sequence
  Synthetic sequence (monoclonal antibody 5.11A partial sequence)
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 19 - protein, 120 aa, Artificial Sequence
  Synthetic sequence (monoclonal antibody 5.11A partial sequence)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

(19) United States
(12) Patent Application Publication
(10) Pub. No.: US 2015/0376278 A1
(43) Pub. Date: Dec. 31, 2015

(54) USE OF A CD28 BINDING PHARMACEUTICAL SUBSTANCE FOR MAKING A PHARMACEUTICAL COMPOSITION WITH DOSE-DEPENDENT EFFECT

(71) Applicant: TheraMAB LLC, Moscow (RU)

(72) Inventors: Thomas Hanke, Wurzburg (DE); Chia-Huey Lin, Wurzburg (DE)

(21) Appl. No.: 14/492,675

(22) Filed: Sep. 22, 2014

Related U.S. Application Data
(63) Continuation of application No. 13/223,439, filed on Sep. 1, 2011, now abandoned.
Application No. 13/223,439 is a continuation of application No. 12/364,395, filed on Feb. 2, 2009, now abandoned.
Application No. 12/364,395 is a continuation of application No. 10/946,836, filed on Sep. 22, 2004, now abandoned.

(30) Foreign Application Priority Data
Sep. 22, 2003 (DE) 10345008.4
Oct. 20, 2003 (DE) 10349371.9

(51) Int. Cl. C07K 16/28

(52) U.S. Cl. CPC C07K 16/2818; C07K 2318/00; C07K 2317/14; A61K 2039/505

(57) ABSTRACT

The invention relates to the use of a CD28-specific superagonistic monoclonal antibody (MAB) or of a mimetic compound of the same, for producing a pharmaceutical composition, wherein the dosage is below or above a defined dosage limit.

### RELATED APPLICATIONS

This application is a continuation of U.S. patent application Ser. No. 12/364,395, filed Feb. 2, 2009, which is a continuation of U.S. application Ser. No. 10/946,836 filed Sep. 22, 2004, which claims priority from DE10345008.4 filed Sep. 22, 2003 and DE10349371.9 filed Oct. 20, 2003.

### SUBJECT OF THE INVENTION

The invention relates to the use of a CD28 binding pharmaceutical substance for making a pharmaceutical composition.

### BACKGROUND OF THE INVENTION AND PRIOR ART

Autoimmunity occurs, when the adaptive immune system composed of B and T lymphocytes (B and T cells) deems autologous structures (“within itself”) heterologous and mobilizes its effector mechanisms against them. Multiple sclerosis, type 1 diabetes and rheumatoid arthritis (RA) are examples of autoimmune diseases, where it is assumed that self-reactive T lymphocytes present in the body play a central role in the pathogenesis of the disease (Ermann and Fathman, 2001).

Normally, T cells are tolerant with regard to autologous tissue and only react on the main histocompatibilty complex (MHC)-mediated detection of heterologous structures with activation and effector functions. This can be explained by different “tolerance-inducing” mechanisms: 1) negative selection: during their maturation in the thymus, T cells having antigen receptors which have a high affinity for autoantigens and are thus potentially autoaggressive and pathogenic are eliminated; 2) anergy induction: in case of incomplete stimulation of T cells, eg by lacking costimulation in secondary lymphoid organs, autoreactive T cells can be brought into a state of temporary functional passivity or anergy; 3) Active suppression: autoreactive and potentially pathogenic T cells are actively suppressed by a specialized T cell subpopulation, so-called regulatory T cells (Tregs, formerly also designated “suppressor cells”) from performing effector functions.

The great importance of this mechanism for maintaining immunologic tolerance is summarized in a multitude of recent survey articles (Maloy and Powrie, 2001; Sakaguchi et al, 2001; Shevach, 2002).

The essential features of the regulatory T cells can be described as follows: i) they are CD4 T lymphocytes having a wide repertoire of T cell antigen receptors (TCR) of the conventional a/13 type Regulatory T lymphocytes maturate in the thymus and are generally characterized in the peripheral lymphoid organs by expression of the low-molecular isoform of the CD45 molecule, the receptors CD25 (IL-2R alpha chain), CD152 (CTLA-4), glucocorticoid-induced TNF receptor family-related protein (GITR), and the transcription factor foxp3 (Khattri et al, 2003). By combined analysis of these markers, a substantial delimitation of regulatory T cells from the remainder of the CD4 T cell population is possible.

By elimination of regulatory T lymphocytes in vivo on the one hand and adoptive cell transfer experiments on the other hand, it could be shown that regulatory T lymphocytes are important for the prevention of organ-specific autoimmune-inflammatory diseases, such as the colitis, the insulitis or orchitis. The ratio in numbers of the regulatory T cells and conventional T cells, which can become pathogenic T cells because of the lacking of Tregs, seems to play an important role (Sakaguchi et al, 1995).

The pharmaceutical mechanism of regulatory T cells is not yet fully clear. The anti-inflammatory cytokines IL-10 and TGFβ seem to play an important role in vivo but not in cell culture (Maloy and Powrie, 2001). Cell culture experiments have demonstrated, on the other hand, that direct contact between the regulatory T lymphocytes and the T lymphocytes to be suppressed is essential; CTLA-4 is thought to be functionally important (Thornton and Shevach, 1998). Even though a complete understanding of the functional mechanisms of regulatory T lymphocytes is still lacking, maintaining immunologic self-tolerance has been shown. Such knowledge can be useful in controlling specific aspects of autoimmunity.

The aim of a pharmacological activation of regulatory T cells for the therapy of autoimmune diseases is to excite the multiplication and function of these cells in the organism, without causing a loss in suppressant function. This approach has proven up to now to be extremely difficult. The activation of regulatory T cells, in the following also called Tregs, via the TCR will, even in combination with a conventional signal via the costimulatory molecule CD28, not normally lead to a proliferation of these cells.

In the rat animal model, it was shown that a novel monoclonal antibody (MAB) with specificity for the CD28 molecule—JJ316—efficiently activates T lymphocytes in vitro as well as in vivo without TCR stimulation (Tacke et al, 1997), i.e. acts “superagonistically.” This antibody—in spite of its strong T cell-stimulatory properties—is very well tolerated in vivo, in contrast to all other known T cell-activating substances (Rodriguez-Palmero et al, 1999; Tacke et al, 1997). Newest results show that with in vivo administration of JJ316, functional Tregs in the rat are overproportionally multiplied (Lin and Hunig, 2003). This preferential stimulation of regulatory T cells with a CD4+CD25+CDTLA-4+ phenotype is most probably the reason for the fair tolerability and the anti-inflammatory effect of the antibody in the organism (see also DE 197 22 888; PCT/DE03/00890)

As a new approach for the multiplication of Tregs for therapeutic purposes in man, a superagonistic antibody for the human CD28 molecule, called CD28-SuperMAB, has been developed. These MABs very efficiently excite in vitro T lymphocytes cells of humans to divide (Luhder et al, 2003). First investigations indicate that, similar to the rat, the CD4+CD25+ regulatory cells present in a low number in the peripheral blood of humans can only with difficulty be multiplied by costimulation, i.e. by anti-TCR plus conventional anti-CD28 stimulation, but very well by superagonistic CD28 stimulation (PCT/DE03/00890). Furthermore, it was shown in a pilot study that an in vitro administration of anti-human CD28-SuperMAB induces in rhesus monkeys in vivo profound activation of T cells, without clinically visible side effects. This indicates that human CD28-SuperMAB may also have an anti-phlogistic effect. Summarizing the above, the tests showed that CD28-SuperMAB, in vitro, activated human Tregs and is very well tolerated in an animal model in vivo, in spite of an induction of T cell activation.

Literature cited above:

Barnes, D A, et al, J Clin Invest 101: 2910-2919, 1998; Ermann, J, et al, Nat Immunol 2:759-761, 2001; Khattri, R, et al, Nat Immunol 4:337-342, 2003; Lin, C H, et al, Eur J Immunol 33:626-638, 2003; Luhder, F, et al, J Exp Med 197:955-966, 2003; Maloy, K J, et al, Nat Immunol 2:816-822, 2001; Rodriguez-Palmero, M, et al, Eur J Immunol 29:3914-3924, 1999.

### Technical Object of the Invention

The invention is based on the technical object of specifying a pharmaceutical composition, which can be used as an anti-inflammatory as well as an immune-reconstituting substance.

### Basics of the Invention and Preferred Embodiments

For achieving the above technical object, the invention teaches the use of a CD28-specific superagonistic monoclonal antibody (MAB) or of a mimetic compound of the same, for producing a pharmaceutical composition for the treatment or prophylaxis of autoimmune-caused inflammatory diseases or for immune reconstitution, wherein the pharmaceutical composition is prepared such that the dosage of the MAB to be administered is below a defined first dosage limit, if the treatment or prophylaxis of an autoimmune-caused inflammatory disease is indicated, and that the dosage of the MAB to be administered is above a defined second dosage limit, if an immune reconstitution is indicated.

The invention is based on a series of new experiments and findings therefrom Such experiments have shown that in the rat model, low doses of JJ316 (superagonistic CD28-specific MAB) expand Tregs to a stronger degree than conventional T cells. Further, a low dosage has in a model for rheumatoid arthritis the same therapeutic effect as a 5 times higher dosage. The basic concept of the invention is to separate the anti-inflammatory effect of the antibody at a low dosage from the generally T cell-stimulating/immune-reconstituting effect at a high dosage. In other words, a pharmaceutical composition is optionally prepared with a dosage of the administration units and/or a dosage instruction, which depending on the desired indication (anti-inflammatory/immune-reconstituting) is below or above the respective defined dosage limits.

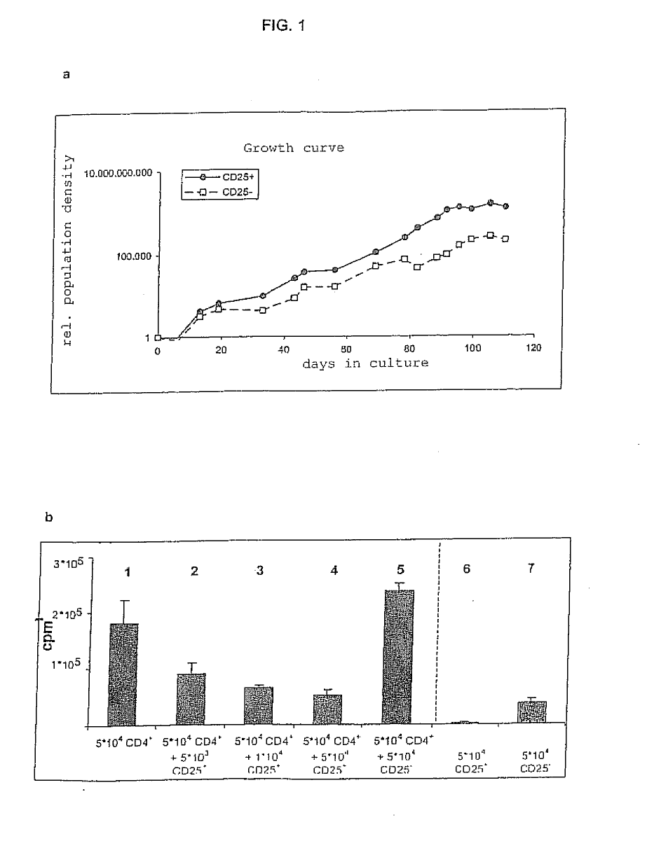

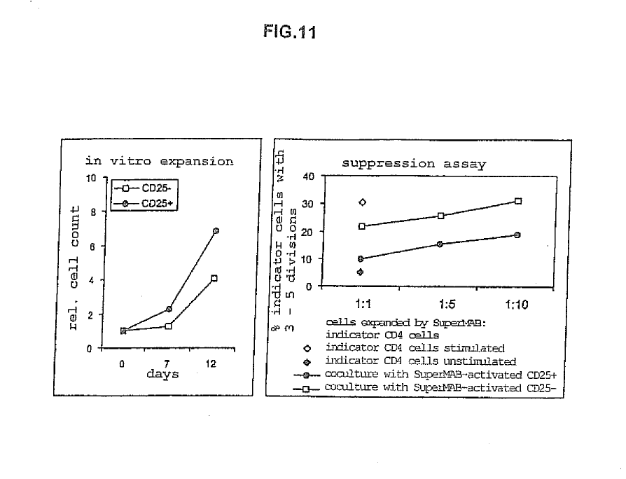

As an aspect of independent importance, it was found that a superagonistic anti-rat or anti-human CD28 antibody permits a long-term culture of functional Tregs in vitro (see also FIGS. 1 and 11), and that therefrom the possibility of a selective expansion of Tregs in vitro and of a therapeutic re-infusion for suppressing autoimmune diseases is generated Insofar, another subject matter of the invention is a method for the in vitro expansion of Tregs, wherein Tregs are incubated with MABs according to the invention and expanded over a period of time of at least 3 to 10 days, preferably by at least the factor 2 to 4 (number of Tregs after the expansion related to number of Tregs before the expansion).

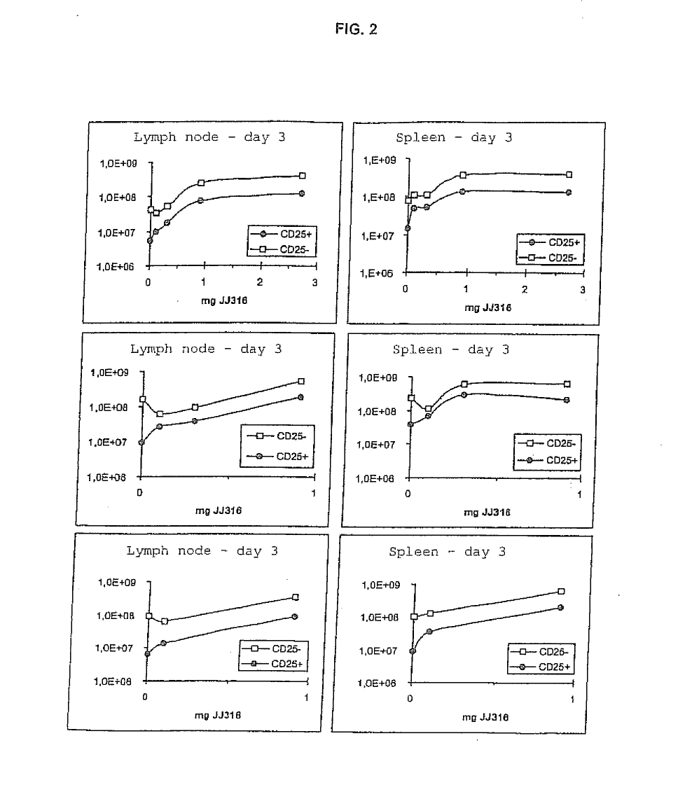

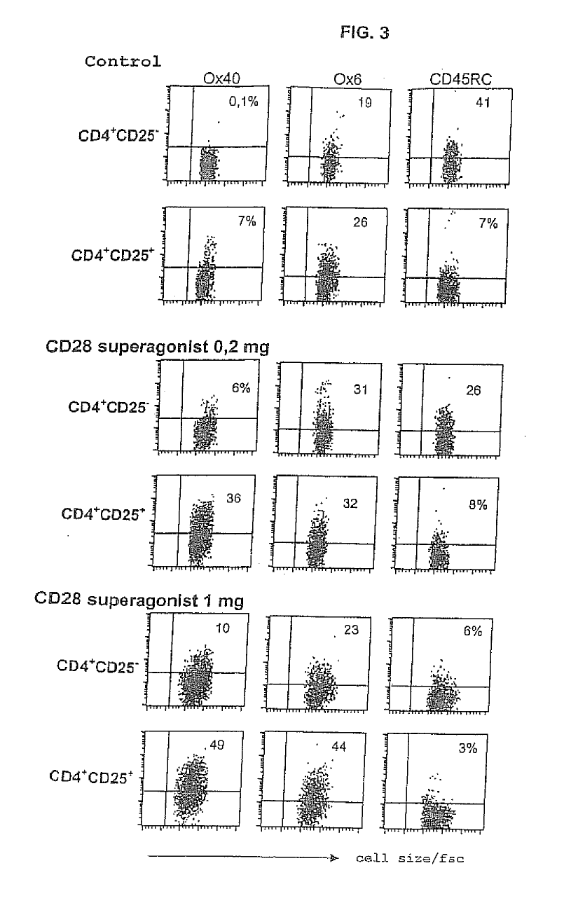

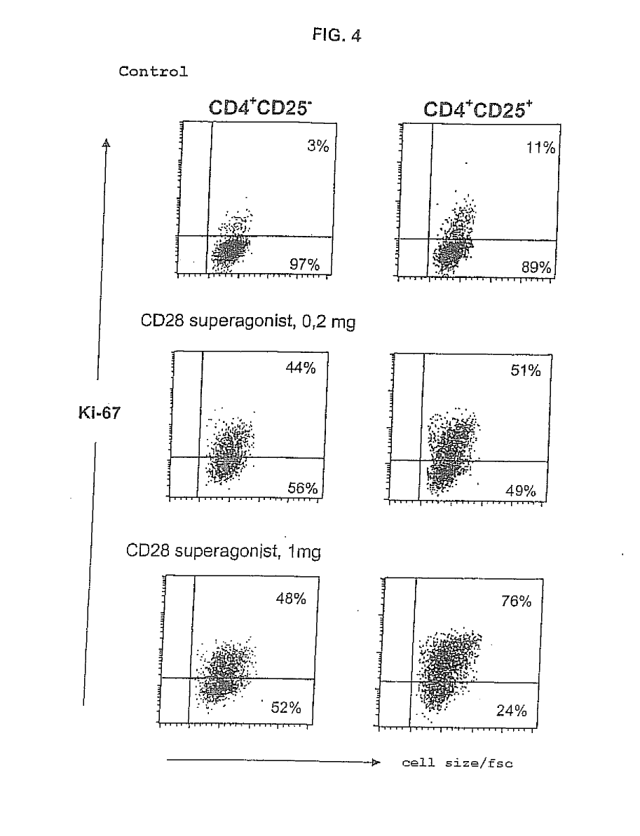

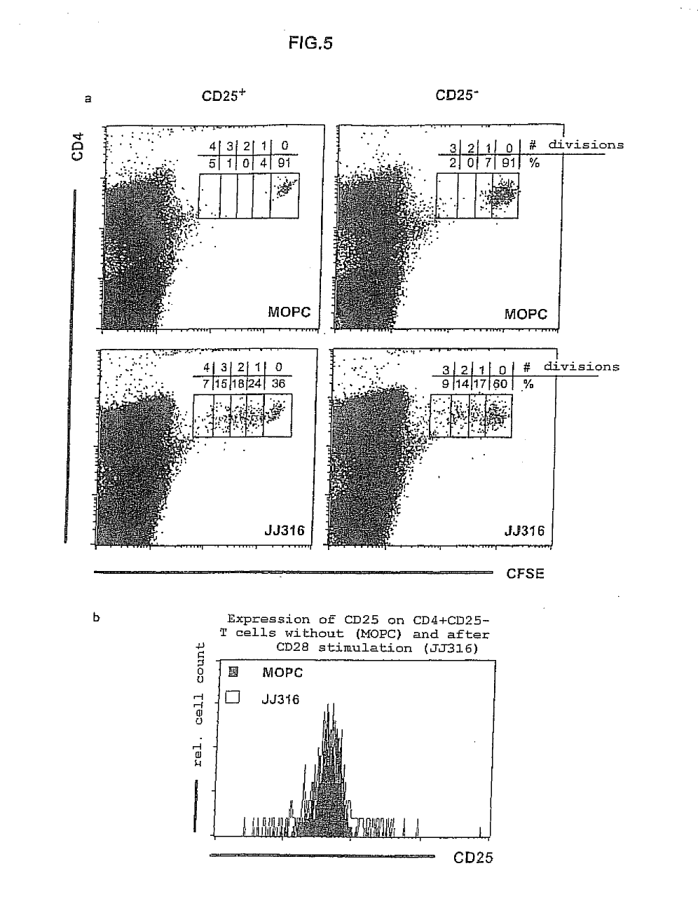

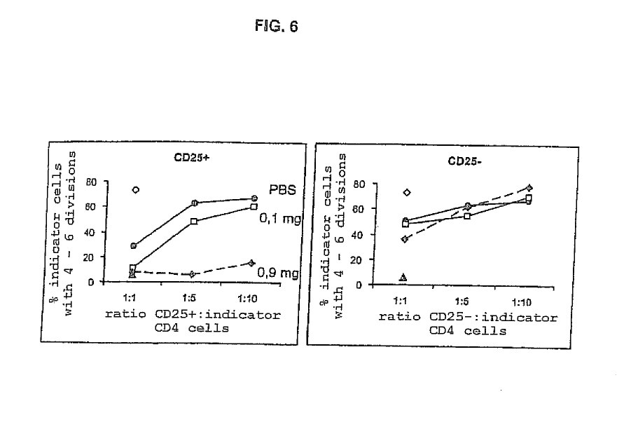

In healthy rats, with lowest-dosage administration of JJ316 (01 to 03 mg/animal), a stronger proliferation of regulatory CD4+CD25+ T cells in lymph nodes and spleen can be found than in conventional CD4+CD25− T cells (see also FIG. 2). These regulatory T cells are phenotypically activated (FIG. 3), and a high portion thereof is in the cell cycle (FIGS. 4, 5). The suppressant properties of the Tregs, measured in vitro in a surrogate assay, are after administration of a lower dosage better than without pre-treatment (FIG. 6).

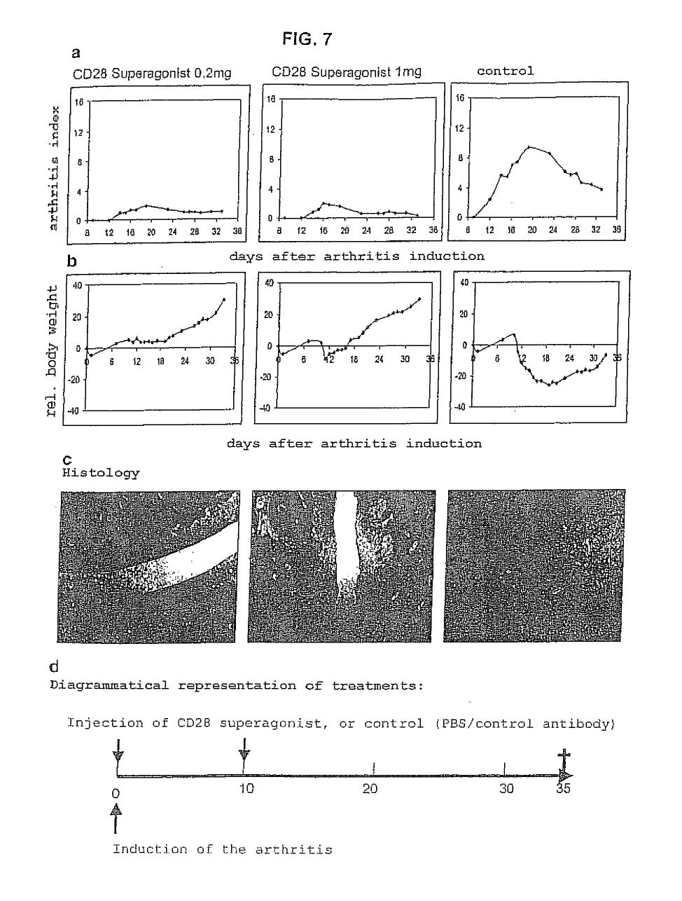

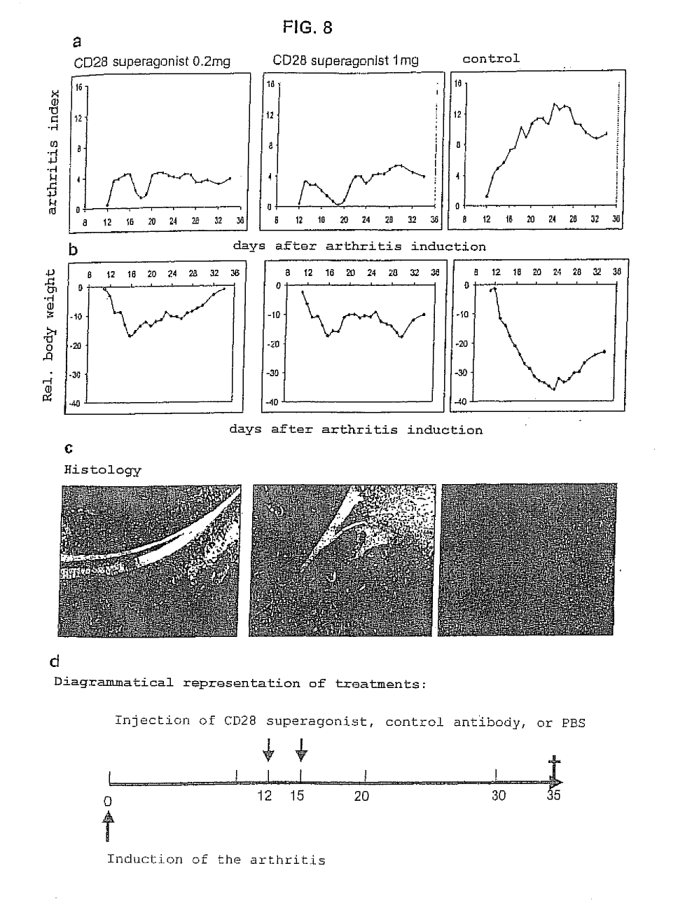

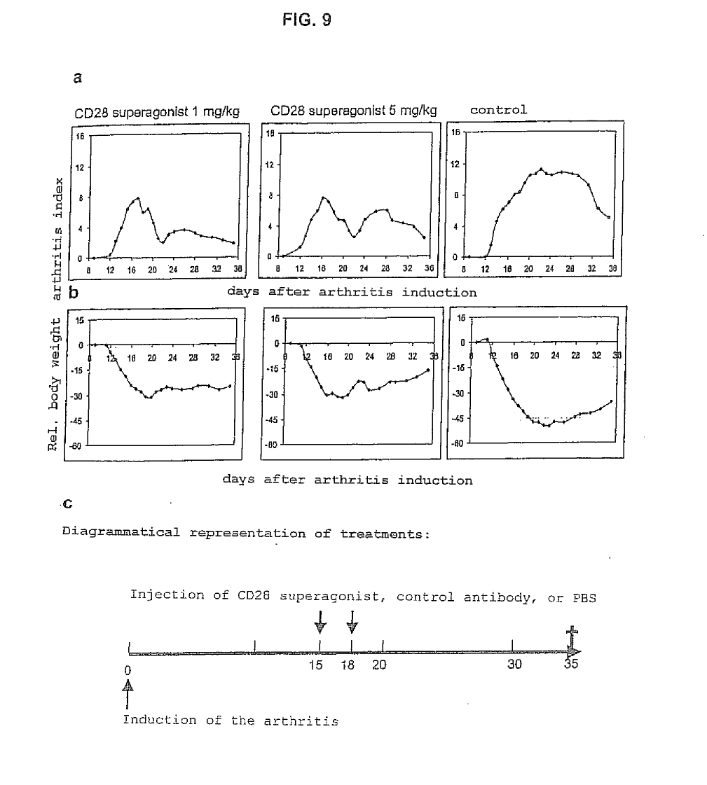

In the Applicant's previous patent application DE 197 22 8887-41, the prophylactic properties of the antibody JJ316 for the prevention of rat-adjuvant arthritis (as an animal model for human rheumatoid arthritis) were mentioned. In the meantime, more comprehensive and better data were collected, which show that this effect cannot only be repeated with more animals and better controls (FIG. 7), but that the antibody administration is therapeutically effective even with an already established disease (FIGS. 8, 9).

All previous experiments concerning the anti-phlogistic properties of the rat CD28 superagonist in the animal models' adjuvant arthritis (DE 197 22 888) and EAN (patent application PCT/DE03/00890) were performed as a routine with, as it became apparent, relatively high antibody doses, namely 1 mg/animal. The new experiments show that at least in adjuvant arthritis, a 5 times lower dosage (02 mg/animal) has the same preventive or therapeutic effect (FIGS. 7-9).

The fact that Tregs preferably proliferate at the low doses, whilst a high dose causes the proliferation of Tregs as well as “normal” T cells, has the implication that the indications of “inflammatory autoimmune diseases” (effective mechanism of the antibody presumably via the activation of Tregs) and “immune reconstitution”, i.e. multiplication of all T cells, can be separated, i e inhibition of inflammation at low dosages and tolerated immune stimulation at high dosages.

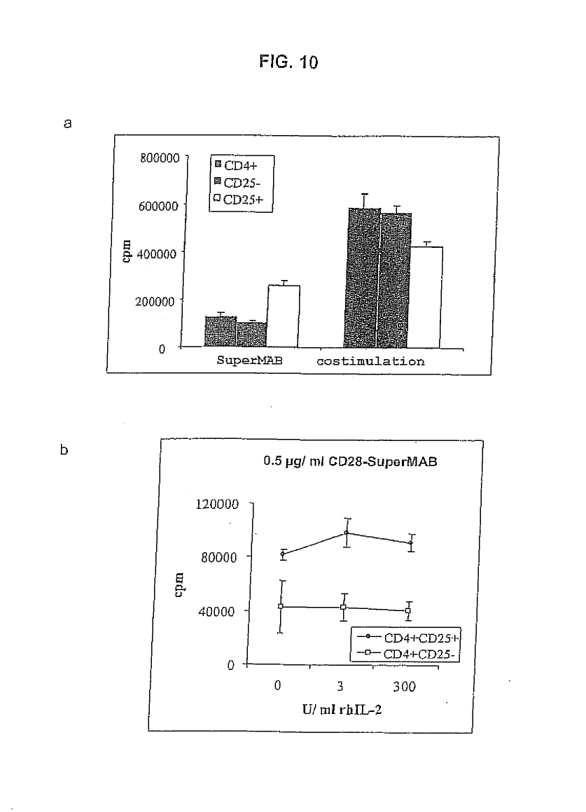

The results of FIG. 10 support older data of the patent application PCT/DE03/00890, namely that the human superagonist can drive Tregs in vitro to proliferation; FIG. 11 shows for the first time that the in vitro CD4+CD25+ cells proliferated also have a suppressor function in an in vitro assay.

It is preferred when the MAB is produced that a non-human mammal is immunized with CD28 or a partial sequence thereof, in particular the C′-D loop and/or spatially adjacent sections, wherein cells are taken from the non-human mammal and hybridoma cells are produced from these cells, and wherein the thus obtained hybridoma cells are selected such that in their culture supernatant there are MABs superagonistically binding to CD28. But other usual methods for obtaining corresponding MABs, such as phage display or human Ig transgenic mice, can be used.

In principle, the antibodies according to the invention may be antibodies derived from the described antibodies, such as chimeric or humanized antibodies.

The mimetic compound is for instance obtainable in a screening method, wherein a prospective mimetic compound or a mixture of prospective mimetic compounds is subjected to a binding assay with CD28 or a partial sequence therefrom, in particular the C′-D loop and/or spatially adjacent sections, and wherein pharmaceutical substances binding to CD28 or to the partial sequence therefrom are selected, possibly followed by an assay for testing for superagonistic stimulation of several to all sub-groups of the T lymphocytes, wherein superagonistically stimulating pharmaceutical substances are selected.

The MAB is, for instance, obtainable from hybridoma cells, as filed under the DSM numbers DSM ACC2531 (MAB: 9D7 or 9D7G3H11) or DSM ACC2530 (MAB: 511A or 511A1C2H3). The MAB or the mimetic compound preferably comprises Seq ID 10, 12, 14, and/or 16, or one or more of the sequences Seq ID 18 and/or 19, or sequences being homologous hereto.

The invention also relates to a method or treatment plan for the treatment or prophylaxis of a disease, wherein either: 1) a pharmaceutical composition is administered to a patient comprising a CD28-specific superagonistic monoclonal antibody or a mimetic compound of the same and in a galenic preparation for a defined and suitable form of administration, for instance intraveneous (IV) injection, or (2) a body liquid is taken from a patient, in particular blood comprising T lymphocytes or precursor cells hereto, and the body liquid, possibly after a processing step, is reacted with a CD28-specific superagonistic monoclonal antibody or a mimetic compound of the same, and the thus treated body liquid is again administered to the patient, for instance by IV injection.

The administered daily dosage for the indication inhibition of inflammation may be below the first dosage limit of 1 mg/kg body weight, or for the indication immune stimulation above the second dosage limit of 2 mg/kg body weight.

An pharmaceutical substance according to the invention binds to CD28 or to a partial sequence therefrom. The partial sequence can for instance contain an amino acid sequence SeqID 1 or 2-7 or 17, which lie at least partially in the region of the C′-D loop of CD28 To one of the sequences with val at the 5′ end, one or more amino acids of the sequence 8 may be connected in the order defined there. The loop is in the region with the sequence GNYSQQLQVYSKTGF. Mimetic compounds according to the invention can be identified in a screening method, a prospective mimetic compound or a mixture of prospective mimetic compounds being subjected to a binding assay with CD28 or a partial sequence therefrom, in particular the C′-D loop, and pharmaceutical substances binding to CD28 or to the partial sequence therefrom being selected, possibly followed by an assay for testing for superagonistic stimulation of several to all sub-groups of the T lymphocytes. In the case of a mixture, it will be suitable to perform a deconvolution. Among the selected mimetic compounds, a “ranking” according to the selectivity and/or affinity may be established, highly affinitive pharmaceutical substances being preferred. In addition to or in lieu of such a ranking, a ranking may be performed according to a quantification of the induction of the Tregs or according to the inhibition of the disease, for instance, in an animal test by using disease models.

An example of a pharmaceutical substance used according to the invention is a superagonistic CD28-specific MAB. It can for instance be produced by immunizing a non-human mammal with CD28 or a peptide comprising a partial sequence therefrom, for instance as mentioned above or homologues hereto. Cells are taken from the non-human mammal cells and hybridoma cells are produced from the cells, and the thus obtained hybridoma cells are selected such that in their culture supernatant, there are MABs binding to CD28. A humanization can be performed with conventional methods Suitable MABs can alternatively be produced by selecting B lymphocytes binding to the loop, and by cloning their expressed immunoglobulin genes An isolation of suitable MABs from phage libraries is also possible.

In detail, a MAB can be obtained from hybridoma cells, as filed under the DSM numbers DSM ACC2531 (MAB: 9D7 or 9D7G3H11) or DSM ACC2530 (MAB: 511A or 511A1C2H3). The MAB may comprise one or more of the sequences Seq ID 10, 12, 14, 16, 18 and/or 19, or sequences being homologous hereto or being (partially) coded thereby. In Seq ID 13, the nucleic acid sequence of the variable region of the heavy chain of an MAB 511A according to the invention is represented. Seq ID 14 shows the peptide coded thereby. Seq ID 15 shows the nucleic acid sequence of the variable region of the light chain of this MAB. Seq ID 16 is the peptide coded hereby. In Seq ID 9 the nucleic acid sequence of the variable region of the light chain of an MAB 9D7 according to the invention is represented. Seq ID 10 shows the peptide coded hereby. Seq ID 11 shows the nucleic acid sequence of the variable region of the heavy chain of this MAB. Seq ID 12 is the peptide coded hereby. Seq ID 18 and 19 show the amino acid sequences of the variable region of a humanized MAB 511A of the light chain and of the heavy chain, respectively.

The invention finally also relates to treatments, wherein to a person suffering from a disease caused by low regulator T cell counts or high T lymphocytes infiltration in organs or tissues, for instance the Guillain-Barré syndrome (GBS) and/or chronic demyelinating polyneuropathy (CDP), a pharmaceutical composition according to the invention is administered in a pharmacologically effective dose and in a galenic preparation suitable for its administration.

### DEFINITIONS

As used herein, “Monoclonal antibodies” (MABs) are antibodies which are produced by hybrid cell lines (so-called hybridomas) which typically have been generated by fusion of a B cell of animal or human origin producing antibodies with a suitable myeloma tumor cell.

The amino acid sequence of human CD28 is known under Accession No NM_{—}006139.

The C′-D loop of CD28 comprises the amino acids 52 to 66 of the above CD28 sequence (for numbering see also Ostrov, D A, et al; Science (2000), 290:816-819). The term C′-D loop will in the following also include any partial sequences therefrom.

A loop or a binding site arranged therein is freely accessible, provided that, for a defined binding partner for the binding site in the loop, there is no steric hindrance by the sequences or molecules following to the loop.

As used herein, “regulatory T cells” are CD4+ T cells inhibiting in a mixture with naïve CD4+ T cells the activation thereof Hereto belong in particular CD4+CD25+ T cells. Another feature of regulatory T cells is, compared to other T cells, a low expression of the high-molecular isoforms of CD45 (human: RA). For regulatory T cells, the constitutive expression of CD152 is typical CD4+CD8-SP thymocytes are one of the essential sources for regulatory T cells. For a further characterization of regulatory T cells, reference is made to the document K J Maloy et al, Nature Immunology, Vol 2, No 9, pages 816 ff, 2001.

As used herein, the “induction of regulatory T cells” is the increase of the metabolic activity, enlargement of the cell volume, synthesis of immunologically important molecules and beginning of cell division (proliferation) upon an external stimulation. As a result, after the induction there are more regulatory T cells than before.

As used herein, “homology” is a sequence identity of at least 70%, preferably at least 80%, most preferably at least 90% on a protein level, a homologous protein or peptide binding a defined binding partner with at least identical affinity. Deviations in the sequence may be deletions, substitutions, insertions and elongations.

As used herein, a “mimetic compound” is a natural or synthetic chemical structure behaving in a defined binding assay as a defined MAB mimicking the mimetic compound.

The term MABs comprises, in addition to structures of the conventional Fab/Fc type, also structures exclusively consisting of the Fab fragment. It is also possible to use the variable region only, the fragment of the heavy chains being connected with the fragment of the light chain in a suitable manner, for instance also by means of synthetic bridge molecules. The term antibody also comprises (possibly complete) chimeric and humanized antibodies.

Superagonistic stimulation of the proliferation of CD28-specific T cells means that no costimulation, i.e. no further binding event in addition to a binding of an MAB or of a mimetic compound is necessary for the stimulation or inhibition of the proliferation, and that several to all sub-groups of the T lymphocytes are activated by such superagonistic CD28-specific MABs.

A lower dosage limit can individually be found out for every pharmaceutical substance according to the invention in that first, the cell counts of CD25+ and CD25-cells per volume unit in the blood of an organism are determined; then, the pharmaceutical substance is administered in respectively increasing doses to the organism or an identical organism, and for instance 3 to 20 days after the administration of a dose; the cell counts of CD4+CD25+ and CD4+CD25− cells per volume unit bloods are again determined; the obtained cell counts (ordinate) versus dosage (abscissa) are entered into a diagram and connected so to form curves for instance by a polynomial fit); and the first derivative of the curves at the dosage is formed and the lower dosage limit is determined as that where the first derivative of the curve for CD4+CD25+ has the same value as the first derivative of the curve for CD4+CD25−, and that within a tolerance deviation of less than 50% (related to the value of the derivative for CD4+CD25+), preferably less than 30%, most preferably less than 10%. This value can simultaneously be the second dosage limit. It may however also be provided that the lower dosage limit is that value, where the value of the derivative for CD4+CD25− changes the sign from negative to positive. The upper dosage limit can then be determined as already mentioned above. Alternatively, the lower dosage limit may be defined by that the relative increase in said period of time of the CD4+CD25+ cells in the organism is at least by a factor two higher than the relative increase of the CD4+CD25− cells (example: quadruplication of the CD4+CD25+ cells at duplication of the CD4+CD25− cells). The upper dosage limit could then be defined as the same relative increase of both cell types. For humans, typical values for the lower dosage limit will be less than 1 mg/kg body weight and day. The upper dosage limit will be 2 mg/kg body weight per day.

Typical diseases of the indication inhibition of inflammation are rheumatoid arthritis, multiple sclerosis, insulin-depending (type 1) diabetes mellitus, Crohn's disease, psoriasis, Guillain-Barré syndrome (GBS), graft-versus-host disease (GVHD).

Typical diseases of the indication immune stimulation are chronic lymphocytic leukemia of the B cell type (B-CLL), acute lymphoblastic leukemia (A-LL), T lymphopenia after chemo- or radio-therapy, as performed for instance with various solid tumors, such as lung carcinoma or mamma carcinoma, HIV infection, and HTLV infection.

### EXAMPLES OF EXECUTION

In the following, the invention and the basic findings are explained in more detail.

FIG. 1 shows the long-term expansion of regulatory CD4+CD25+ T cells in vitro. It has been shown by Lin et at (Lin and Hunig, 2003) that the superagonistic anti-rat CD28 MAB JJ316 is capable of very efficiently activating regulatory CD4+CD25+CTLA-4+ T cells in short-term cell culture. It was further explored whether the long-term expansion of Tregs was another achievable outcome. FIG. 1a shows that this antibody does indeed allow a long-term expansion of the CD4+CD25+ T cells: 6×10̂4 CD4+CD25+ and CD4+CD25− T cells were isolated from a normal Lewis rat and cultivated in presence of JJ316 (5 μg/ml) and interleukin-2 (300 U/ml) in microtiter plates (96-well, 12-well or 6-well plates) at 37° C., and the cells were weekly supplied with fresh reagents. Also in this long-term culture, a preferential expansion of the CD4+CD25+ T cells compared to the CD4+CD25− T cells can be observed Beginning from the initial cell count (FIG. 1a shows relative values with regard to the cell count; the absolute cell count on day 0 was taken as 1), the CD4+CD25+ T cells could by multiplied by the factor 61×10̂7. For the CD4+CD25− T cells, this value is only 67×10̂5 (FIG. 1a).

The functional properties of regulatory T cells in vitro are characterized by the fact that they are capable of suppressing the proliferation of conventional T cells. By means of an in vitro test system, it was investigated whether the CD4+CD25+ T cells expanded by JJ316 could maintain this function (FIG. 1b): 5×10̂4 conventional CD4+ T cells serving as indicator cells in this test were cultivated either alone or together with increasing amounts (5×10̂3, 1×10̂4 or 5×10̂4 cells) of the CD4+CD25+ or CD4+CD25− T cells of day 78 of the long-term culture (FIG. 1a) for three days in 96-well microtiter plates in 02 μl. The stimulation of the cells was made by the addition of lectin ConA (2 μl/ml) and antigen-presenting cells (5×10̂4). The cell proliferation in the culture was measured by means of integration of 3H thymidine during the last 16 hours of the assay, as described in the literature. The polyclonal stimulation of CD4+ indicator cells by ConA (column 1) could significantly be reduced by the addition of the multiplied CD4+CD25+ T cells (columns 2-4). In contrast, the proliferated CD4+CD25− T cells (column 5) showed no suppressant functions, as expected In contrast to the CD4+CD25− T cells (column 7), the JJ316-stimulated CD4+CD25+ T cells (column 6) could be stimulated by ConA to a very low degree only, thereby indicating that the values shown in columns 2-4 nearly exclusively reflect the proliferation of the indicator cells. This result shows that the in vitro expanded regulatory T cells could maintain their functional—i.e. suppressant—properties.

FIG. 2 shows the preferential multiplication of regulatory CD4+CD25+ T cells after in vivo application of JJ316 in normal Lewis rats. After the above in vitro experiments had shown that superagonistic CD28-specific MABs can induce a preferential expansion of CD4+CD25+ T cells while maintaining their functional properties, it was investigated whether similar effects can be observed also after injection of these antibodies in the test animal: normal adult Lewis rats were JJ 316 IV applied in different doses (01; 03 and 09 and 27 mg/animal). After 3 days, spleen and lymph nodes were removed, and the lymphocyte subpopulations of these organs were analyzed in the FACS (measured parameters: CD5, CD4 and CD25). The relative values of the CD4+CD25+ and the CD4+CD25− T cells determined by the FACS analysis were multiplied with the total cell count/organ, and the thus obtained number of the two populations were recorded as a function of the applied dosage. In FIG. 2, the results of three independent experiments are represented. As evidenced by the slope of the curve, particularly in the lower dosage range and in the lymph nodes, an increased expansion of CD4+CD25+ compared to CD4+CD25− T cells was observed after in vivo application of JJ316. This experiment clearly shows that superagonistic CD28-specific monoclonal antibodies multiply mainly regulatory T cells in vivo at low doses and can thus potentially re-establish the disturbed balance for many autoimmune diseases between regulatory T cells and autoreactive T cells.

FIG. 3 shows the phenotype of JJ316-expanded cells In the previous experiment, the absolute cell counts of CD4+CD25+ and CD4+CD25− T cells were determined after in vivo applications, and it was shown that for the lower dosage JJ316, the number of the CD4+CD25+ cells in the lymph node increases to a higher degree than the number of the CD4+CD25− cells. Then, it was of interest to determine the activation or differentiation state of the CD4+CD25+ and CD4+CD25− T cells by means of flow cytometric analysis of the expression of the T cell markers OX40, OX6 (MHC class II) and CD45RC 02 or 1 mg JJ316 were IV injected into male rats Four days later, i.e. at a time near to the maximum expansion of the CD4+CD25+ cells (Lin and Hunig, 2003), lymph node cells were isolated, and the cell surface expression of OX40, OX6 (MHC class II) and CD45RC was determined FIG. 3 shows the relative cell sizes (x axis) relative to the activation markers (y axis) among the CD4+CD25− and CD4+CD25+ cells of the control group (top), the low-dosage group (center) and the high-dosage group (bottom). High expression of OX40 or OX6 and low expression of CD45RC and increase of the relative cell size in the so-called forward scatter, fsc, characterizes activated cells. Therefore, CD4+CD25− as well as CD4+CD25+ cells were activated by both doses of JJ316. For the conventional CD4+CD25− cells, the activation seemed to be stronger for the higher dosages than for the lower dosages (increase, in particular, of the fsc and reduction of CD45RC; however, lower increase of OX6 was observed). The regulatory CD4+CD25+ cells, had, as expected in the control group, a high portion of pre-activated cells (low portion of CD45RC+ cells), which was increased by both doses (see in particular fsc, OX40). The higher expression and the larger fsc indicate that the activation in the high-dosage group was stronger than in the low-dosage group.

FIG. 4 shows the in vivo proliferation of CD4+CD25+ and CD4+CD25− T cells after administration of JJ316 FIG. 3 showed that conventional CD4+CD25− cells as well as regulatory CD4+CD25+ cells were phenotypically activated in vivo by both doses of JJ316. Subsequently, by means of the proliferation marker Ki67, it was measured whether the activation also correlates with cell division. The test approach was identical to that of FIG. 3. The portion of the CD4+CD25+ or CD4+CD25− cells being in the cell cycle was determined by extracellular staining on CD4 and CD25 expression as well as by intracellular staining on Ki-67 expression (characterizes cells, which are in the S or G2/M phase of cell division and thus actively proliferating). In FIG. 4 can be seen that with regard to the control group, the treatment with JJ316 induces in both populations a strong Ki-67 expression and thus active DNA synthesis, which was slightly stronger for the high doses than for the low doses. As a summary, the experiments show that by IV application of 02 mg or 1 mg JJ316, a profound phenotypic activation and multiplication of Tregs in rats is induced.

FIG. 5 shows that JJ316 expands in vivo CD4+CD25+ T cells, without inducing the expression of CD25 to before CD25-negative (conventional) T cells. The prior experiments could not provide direct proof that the increased absolute number of the CD4+CD25+ T cells after the in vivo application of JJ316 is the consequence of the expansion of these cells and does not rely on that originally CD25-negative T cells express after activation of this protein as an “activation marker” on the surface (as this can be observed after in vitro activation of T cells). For clarifying this question, CD4+CD25+/CD4+CD25− T cells from the spleen and lymph nodes of normal rats were isolated, and the two populations were labeled with the dye carboxyfluorescein succinimidyl ester (CFSE, 25 μM) CFSE was incorporated in the cytoplasm of the cell and distributed after every cell division by 50% each on the daughter cells. By means of FACS analysis, the CFSE content of a cell population can be analyzed and thus the number of the obtained cell divisions can be determined 5×10̂6 CD4+CD25− T cells and 1×10̂6 CD4+CD25+CFSE-labeled T cells were transferred into respectively different receiver animals, into which one day later either a control antibody (MOPC) or JJ316 each in the low doses of 02 mg/animal were IV applied. After two days, spleen and lymph nodes of these animals were removed, the T cells were purified by means of nylon wool, and their CFSE content were analyzed in the FACS FIG. 5a shows that the application of JJ316 not only induces a dilution of the CFSE content of the transferred CD4+CD25−, but also of the CD4+CD25+ T cells. This result thus supplies direct proof that JJ316 can induce in vivo an expansion of CD4+CD25+ T cells. In this experiment, too, we could observe a preferential expansion of the CD4+CD25+ T cells by JJ316, since the average number of cell divisions is higher (13) for the CD4+CD25+ T cells than for the CD4+CD25− T cells (07). This finding that CD25 is not induced in vivo in CD4+CD25− T cells by JJ316 stimulation (FIG. 5b), further indicates that the increased number of CD4+CD25+ T cells is exclusively a consequence of the expansion of CD4+CD25+ “mother” cells.

FIG. 6 shows the functional characterization of regulatory CD4+CD25+ T cells expanded in vivo by JJ316. It is of decisive importance whether the CD4+CD25+ T cells expanded in vivo by JJ316 maintain their suppressant properties. For clarifying this question, JJ316 was applied in different doses (01 mg and 09 mg) into normal rats, as described above. After three days CD4+CD25+/CD4+CD25− T cells were isolated and tested in vitro for their suppressant potential: it was investigated how strongly the two populations are capable of inhibiting in vitro the proliferation of CD4+ T cells activated by conventional costimulation (=indicator cells). The expanded CD4+CD25+ or CD4+CD25− T cells were cocultivated in this test in different ratios (1:1, 1:5 and 1:10) with CFSE-labeled CD4+ indicator cells in presence of monoclonal antibodies with specificity for the T cell receptor (R73; 1 μg/ml) and CD28 (JJ319; 05 μg/ml) for five days, and then the CFSE content of the indicator cells (as a measure of the occurred cell divisions) was analyzed in the FACS. Whereas in the absence of the CD4+CD25+ T cells, still approximately 75% of the indicator cells could undergo 4-6 cell divisions (FIG. 6, open rhombus), this portion was distinctly reduced after addition of the expanded CD4+CD25+ T cells, and that the stronger, the higher was the dosage of JJ316 previously applied in the animals (FIG. 6, closed circles=PBS control, open squares=01 mg JJ316, closed rhombi=09 mg JJ316). On the other hand, such an effect could not be observed for the CD4+CD25− T cells. After in vivo application of superagonistic CD28-specific monoclonal antibodies, thus not only a purely numerical multiplication of regulatory T cells is induced, rather the suppressant properties of these cells can even be increased with an increasing antibody dosage.

FIG. 7 shows the preventive effect of JJ316 on the development of adjuvant arthritis. The previous results show that JJ316 induces in vivo and in vitro an expansion of functional regulatory CD4+CD25+ cells. The important question was open whether this expansion also correlates with an anti-inflammatory effect of the antibody, and if that is the case whether the Tregs are really causally involved in the mediation of such an effect. Whilst the following results indicate a clearly positive answer to the first question, the question for the mechanism of the inflammation prophylaxis and therapy, as shown by FIGS. 7-9, by CD28 superagonists is not yet fully answered. For the investigation of the anti-phlogistic effect of JJ316, the animal model of the adjuvant arthritis in Lewis rats (Barnes et al, 1998) was used For the disease induction, 6-8 weeks old male rats (200 to 250 g) were intradermally injected (day 0) at the base of the tail with 01 ml Mycobacterium tuberculosis (heat-killed) in IFA (incomplete Freund's adjuvant, 5 mg/ml). In order to investigate in the first group the prophylactic effect of JJ316, six rats each were intravenously applied on day 0 and day 10 02 or 1 mg JJ316. As a control group served six rats, which were treated on day 0 and day 10 with 0 mg, 02 mg or 1 mg irrelevant control antibodies of the same isotype (mouse IgG1) (shown is here as in FIGS. 8 and 9 a group treated with 1 mg control antibody; identical results were obtained in the other control groups). The development of arthritis was continuously examined by objective evaluation of the joint inflammations, represented as the arthritis index consisting of a combined determination of reddening, swelling and stiffening of the joints, and the surrogate parameter “weight measurement.” The experiment was terminated on day 35 by killing the animals. The back paws were fixed in a 10% formalin solution for 72 hours. After decalcification in 10% EDTA solution (24 weeks), the joint tissue was histologically processed as described by (Barnes et al, 1998) by staining with safranin O, fast green and hematoxylin and analyzed with regard to cartilage destruction and cellular infiltrates. In FIG. 7a, b the result of the preventive treatment of six animals each are summarized in their mean values. The results indicate that in the control animals, arthritis occurs beginning on day 9 with a maximum occurrence after approximately 3 weeks (top right), which is characterized by an accompanying weight loss (center right). By treatment with a low dose of 02 mg JJ316 (left column), same as with a high dose of 1 mg JJ316 (central column), the development of the arthritis as well as the weight loss could nearly completely be prevented. It is remarkable that the prophylactic effect of the low dosage is at least as good as the effect of the 5-fold higher dosage. The equivalent protection from arthritis by a high- and low-dosage JJ316 treatment could also be shown on a histologic level: whilst for control animals, the joints of the back paws are characterized by massive cellular infiltrates as a sign of an inflammation reaction (FIG. 7c, right), the joint gaps of the JJ316-treated animals have a normal architecture (FIG. 7c, center and left). The treatment scheme is summarized in FIG. 7d. In summary, these results show that high- and low-dosage JJ316 treatment at the time of the arthritis induction has an equally strong preventive effect on the development of the arthritis.

FIG. 8 shows the therapeutic effect of JJ316-treatment on adjuvant arthritis (early intervention). Then the important question was examined whether JJ316 treatment has not only a preventive, but also a curative effect on the disease progress. This is particularly relevant, since for the clinical treatment of rheumatoid arthritis with human CD28-SuperMAB, no prophylaxis, but rather, only therapy is possible. In the experiment shown in FIG. 8, arthritis was induced for six animals each per group, as described in FIG. 7 On day 12 and 15 after disease induction, i.e. at the time of a very mild development of the symptoms (average arthritis index 05) and three days later, the animals were treated with 02 mg JJ316, 10 mg JJ316 or an irrelevant control antibody. As is shown in FIG. 8a, for the JJ316-treated animals there was, after an initial development of the symptoms, a stabilization of the disease (max arthritis index 4-5), and in the meantime a distinct reduction of the symptoms and a stabilization of the weight. Again the therapeutic effect was very similar for high and low doses of JJ316. For the control-treated animals, there was however a fulminant arthritis with a maximum arthritis index of >12 and a progressive weight loss (FIG. 8a, b). Equally, the JJ316 treatment led with both doses to a maintenance of the normal architecture of the joint tissue, which in the control group showed clear signs of inflammatory infiltrates and morphologic destruction (FIG. 8c). In summary, with early therapeutic intervention, a low dosage of JJ316 as well as a high dosage of JJ316, will prevent the clinical development of a serious arthritis and the destruction of tissue.

FIG. 9 shows the therapeutic effect of JJ316 treatment on adjuvant arthritis (late intervention). Subsequently, it was investigated whether an arthritis therapy is possible by CD28 superagonist administration even with clearly developed disease symptoms. The experimental approach corresponded to that as described in FIG. 8; however, animals that were already clearly diseased, were treated on day 15, i.e. at a time when the arthritis development was given the mark 65 on average, and on day 18 with JJ316 (02 and 10 mg/animal) or with a control antibody (FIG. 9). For the JJ316-treated animals, a distinct reduction in the disease symptoms approximately beginning on day 16 was found. This effect was even more developed for a low JJ316 dosage than for the high dosage. Further, the weight was stable for the experimental group, whereas the control group had a progressing weight loss (FIG. 8b). In summary, even in this extremely complicated late pharmacological intervention, a low JJ316 dosage had an at least equally clear curative effect on the course of the arthritis in the rat model as a high JJ316 dosage.

FIG. 10 shows the preferential expansion of human CD4+CD25+ T cells by means of SuperMAB. As already described in earlier documents, superagonistic monoclonal antibodies with specificity for human CD28 (=SuperMAB) were generated. Here, too, it was important to optimize the stimulation and culture conditions, in order to guarantee an efficient expansion of human CD4+CD25+ T cells by means of SuperMAB: from voluntary healthy donors, T lymphocytes were prepared and separated into “conventional” CD4+CD25− and regulatory CD4+CD25+ T cells (purity as a routine approximately 90%) 5×10̂4 each of both populations were cultivated in the presence of cross-linked (by means of 40 μg/ml anti-mouse Ig antiserum) SuperMAB (10 μg/ml) or by classic costimulation by means of cross-linked monoclonal antibodies with specificity for CD3 (10 μg/ml) and CD28 (10 μg/ml) in 96-well microtiter plates. After three days, the proliferation was tested by means of incorporation of 3H thymidine. Similarly, as was already described for JJ316, CD4+CD25+ T cells were also driven by SuperMAB to a stronger degree into proliferation than conventional CD4+CD25− T cells (FIG. 10a). This result was reproduced in a following experiment, wherein simultaneously the effect of the growth factor interleukin-2 was investigated for the stimulation of the CD4 subpopulations CD4+CD25+ and CD4+CD25− by means of SuperMAB As shown in FIG. 10b, IL-2 did not have any decisive influence in this experiment on the proliferation of phenotypically regulatory CD4+CD25+ or conventional CD4+CD25− T cells. These in vitro experiments show that SuperMAB is also capable of primarily multiplying regulatory T cells.

FIG. 11 verifies that CD4+CD25+ T cells expanded in vitro by SuperMAB are regulatory T cells. As already explained for the system rat, here the question was, too, whether the CD4+CD25+ T cells expanded by SuperMAB could maintain their functional properties, namely the capability to suppress the proliferation of conventional T cells For this purpose, CD4+CD25+ and CD4+CD25− T cells of healthy donors were isolated and cultivated for 12 days in presence of SuperMAB (04 μg/ml) and interleukin-2 (100 U/ml). In this experiment, too, the CD4+CD25+ T cells could be driven into the proliferation better than the CD4+CD25− T cells (FIG. 11a). In a second culture, then the cells expanded by SuperMAB were tested for their suppressant potential. Analogously to the experiments projected for the rat, it was investigated whether they are capable of inhibiting the proliferation of syngeneic and CFSE-labeled CD4+ T cells activated by conventional costimulation (=indicator cells): the expanded CD4+CD25+ and CD4+CD25− T cells were cocultivated for five days in different ratios (1:1, 1:5 and 1:10) with CFSE-labeled CD4+ indicator cells in the presence of monoclonal antibodies with a specificity for CD3 (01 μg/ml) and CD28 (005 μg/ml), and then the CFSE content of the indicator cells (as a measure of the achieved cell divisions) was analyzed in the FACS. Whilst in absence of the CD4+CD25+ T cells, still approximately 30% of the indicator cells could undergo 3-5 cell divisions (FIG. 11b, open rhombus), this portion was clearly reduced after addition of the SuperMAB-expanded CD4+CD25+ T cells (closed circles). Also, a low suppressant effect showed the expanded CD4+CD25− T cells, too (open squares), which possibly is caused by the fact that a few CD25+ T cells were present within the population of CD25− T cells and they have transferred their suppressant properties to a part of the CD25− T cells during the SuperMAB-driven expansion phase. Alternatively, a stimulation of CD25-regulatory T cells by SuperMAB is also a possibility. This experiment shows that, as has already been shown for the rat system, SuperMAB-expanded human CD4+CD25+ T cells also maintain their functional properties.

1. A method for the treatment of arthritis in a patient in need thereof, comprising administering a pharmaceutical composition comprising a CD28-specific superagonistic monoclonal antibody (MAB) or a mimetic compound of the same.

2. The method of claim 1, wherein the MAB is prepared from a non-human mammal immunized with CD28 or a partial sequence thereof

3. The method of claim 1, wherein the mimetic compound is obtainable in a screening method comprising the following steps: a prospective mimetic compound or a mixture of prospective mimetic compounds is subjected to a binding assay with CD28 or a partial sequence therefrom, and pharmaceutical substances binding to CD28 or to the partial sequence thereof are selected.

4. The method of claim 1, wherein the MAB is obtainable from hybridoma cells, as filed under the DSM numbers DSM ACC2531 (MAB: 9D7 Or 9D7G3H11) or DSM ACC2530 (MAB: 511A or 511A1C2H3).

5. The method of claim 1, wherein the MAB or the mimetic compound comprises one or more of the sequences SEQ ID NOS: 10, 12, 14, or 16, or one or more of the sequences SEQ ID NOS: 18 or 19, or sequences being homologous hereto.

6. The method of claim 1, wherein the pharmaceutical composition is a galenic preparation for a defined and suitable form of administration comprising intravenous injection.

7. (canceled)

8. The method of claim 2, wherein the partial sequence of CD28 comprises a C′-D loop or spatially adjacent sections of CD28.

9. The method of claim 2, wherein the MAB is further prepared by taking cells from a non-human mammal and producing hybridoma cells from the non-human mammal cells, wherein the hybridoma cells are selected such that their culture supernatant contains MABs which superagnonistically bind to CD 28.

10. The method of claim 3, wherein the partial sequence of CD28 comprises a C′-D loop, or spatially adjacent sections of CD28.

11. The method of claim 3, further comprising the following step: the pharmaceutical substances binding to CD28 or to the partial sequence thereof is assayed to test for superagonistic stimulation of several to all sub-groups of the T lymphocytes, wherein superagonistically stimulating pharmaceutical substances are selected.

12. A method for the treatment of arthritis in a patient in need thereof, wherein from the patient is taken a body liquid, comprising blood comprising T lymphocytes or precursor cells hereto, and the body liquid is reacted with a CD28-specific superagonistic monoclonal antibody or a mimetic compound of the same, and the thus treated body liquid is again administered to the patient by a suitable form of administration comprising intravenous injection.

13. The method of claim 1, wherein the dosage of the MAB to be administered is below 1 to 10 mg/kg body weight.

14. The method of claim 13, wherein the dosage of the MAB to be administered is below 1 mg/kg body weight.

15. The method of claim 6, wherein the dosage of the MAB to be administered is below 1 to 10 mg/kg body weight.

16. The method of claim 15, wherein the dosage of the MAB to be administered is below 1 mg/kg body weight.
